(19) United States
(12) Patent Application Publication
(10) Pub. No.: US 2015/0140595 A1
(43) Pub. Date: May 21, 2015

(54) FORMULATIONS FOR THE SYNTHESIS OF PARAMAGNETIC PARTICLES AND METHODS THAT UTILIZE THE PARTICLES FOR BIOCHEMICAL APPLICATIONS

(71) Applicant: Joseph Gerard UTERMOHLEN, (US)

(72) Inventor: Joseph Gerard Utermohlen, Tucson, AZ (US)

(21) Appl. No.: 14/403,041

(22) PCT Filed: May 22, 2013
(86) PCT No.: PCT/US13/42263

§ 371 Date: Nov. 21, 2014

Related U.S. Application Data
(60) Provisional application No. 61/650,245, filed on May 22, 2012.

(51) Int. Cl. C12N 15/10; H01F 1/01; C01G 53/00; C01G 49/00; C01G 51/00

(52) U.S. Cl. CPC C12N 15/1013; C01G 49/0018; C01G 49/0027; C01G 49/0036; C01G 49/0045; C01G 49/0063; C01G 49/0072; C01G 51/40; C01G 53/40; H01F 1/01; C01P 2006/42
USPC 435/29; 536/25.41; 530/415; 435/270; 423/594.1; 423/594.2; 252/62.54

(57) ABSTRACT

A set of paramagnetic particles synthesized by co-precipitation methods wherein an alkaline hydroxide solution is mixed with a metal salt solution. The alkaline hydroxide features ammonium hydroxide, potassium hydroxide, sodium hydroxide, or mixtures thereof. The metal salt solution features at least one ferrous salt and at least one tetravalent metal salt selected from Group 4 elements of the Periodic Table. The concentration of the ferrous salt is equal to or greater than the concentration of the tetravalent metal salt. The paramagnetic particles may be used for bioprocessing via magnetic fields. Bioprocessing, for example, may include purifying, concentrating, or detecting biomolecules of interest (e.g., nucleic acids, carbohydrates, peptides, proteins, other organic molecules, cells, organelles, microorganisms, viruses, etc.), or other magnetic field-based processes common to applications in separation science, diagnostics, molecular biology, protein chemistry, and clinical practice.

### CROSS REFERENCE

This application claims priority to U.S. provisional application Ser. No. 61/650,245 filed May 22, 2012, the specification of which is incorporated herein by reference in its entirety.

### FIELD OF THE INVENTION

The invention relates to paramagnetic particles synthesized from metal salt solutions (e.g., via co-precipitation methods). The paramagnetic particles may be used for bioprocessing via magnetic fields. Bioprocessing, for example, may include purifying, concentrating, or detecting biomolecules of interest (e.g., nucleic acids, carbohydrates, peptides, proteins, other organic molecules, cells, organelles, microorganisms, viruses, etc.), or other magnetic field-based processes common to applications in separation science, diagnostics, molecular biology, protein chemistry, and clinical practice. The metal salt formulations used for paramagnetic particle synthesis described herein may be selected to be chemically inert. The metal salt formulations and paramagnetic particles may be used for enzymatic assays, DNA amplification, RNA synthesis, or immunoglobulin-based assays, detection chemistry based on enzymatic conversion of dyes to detectable forms, or any other appropriate assay.

### BACKGROUND OF THE INVENTION

Numerous biological applications of magnetic separation methods have been described for isolating or purifying cells (e.g., prokaryotes, eukaryotes, viruses) and for applications based on manipulation of biomolecules, e.g., diagnostic and detection testing. Examples of such applications are described in following patents: U.S. Pat. No. 3,470,067, U.S. Pat. No. 3,970,518, U.S. Pat. No. 4,675,113, U.S. Pat. No. 4,935,147, U.S. Pat. No. 5,108,933, U.S. Pat. No. 5,167,811, U.S. Pat. No. 5,320,944, U.S. Pat. No. 5,523,231, U.S. Pat. No. 5,665,554, U.S. Pat. No. 5,705,628, U.S. Pat. No. 5,736,349, U.S. Pat. No. 5,898,071, U.S. Pat. No. 5,945,525, U.S. Pat. No. 6,027,945, U.S. Pat. No. 6,368,800, U.S. Pat. No. 6,620,627, and U.S. Pat. No. 6,936,414, the disclosures of which are incorporated herein in their entirety.

Most the paramagnetic solid supports used for magnetic separation of cells and biomolecules are beads of micron dimensions, ranging in size from about 1.0 μm to about 100 μm in diameter. These beads are composed of diamagnetic material, such as plastics or silica, which coat or encapsulate paramagnetic iron oxide as ferrite nanoparticles. This bead structure can help ferrite from exposure to water and oxygen. For these beads, the most common ferrites are hematite (Fe2O3), an iron (HI) oxide, magnetite (Fe3O4), a spinel ferrite composed of iron oxide a crystalline matrix composed of ferrous oxide and ferric oxides. In addition to these iron oxides, mixed metals ferrites are also used for bio-separation applications. These include cobalt ferrite (CoFe2O4), nickel ferrite (NiFe2O4), or zinc ferrite (ZnFe2O4). For all of these ferrites, like magnetite ferrites, there is a general need to sequester the mixed ferrite particle from water exposure.

The most common types of water-impermeable coatings or encapsulating media are cross-linked agar or alginate, graphene or graphite, latex, polystyrene, silica, silane, silicones, or mixtures thereof. Examples of such media as commercial paramagnetic beads are MagJet Beads, Dynal MyOne Bead, Ampure Beads, and MagneSil. The following patents describe paramagnetic solid supports that represent the current art: U.S. Pat. No. 4,628,037, U.S. Pat. No. 4,654,267, U.S. Pat. No. 4,672,040, U.S. Pat. No. 4,795,698, U.S. Pat. No. 5,206,159, U.S. Pat. No. 5,451,245, U.S. Pat. No. 5,648,124 and U.S. Pat. No. 6,767,635, the disclosures of which are incorporated herein in their entirety.

In some embodiments, a property of the particles of the present invention is a degree of paramagnetism that is strong enough to be useful for magnetic separation. In some embodiments, the particles of the present invention have surface stability in water. In some embodiments, the particles of the present invention have biochemical compatibility with most enzyme-based assays. In some embodiments, the particles of the present invention have chemical stability to the majority of reagents used for peptide synthesis, protein processing, nucleic acid isolation, and for diagnostic assays.

Metal oxides in the form of clay, aluminum oxide, and iron oxides such as magnetite have all been reported to be useful solid supports for biological chromatography. For example, the use of colloidal-size bentonite particles as the solid support for purifying enzymes was described by G. Alderton, W. H. Ward, and H. L. Fevold (1945) Journal of Biological Chemistry 157: 43-58. The surface of the bentonite clay had affinity properties toward lysozyme and was used to purify the enzyme from other egg proteins. Clay processing was based on centrifugation.

DNA purification processes that utilize magnetic separation based on solid support composed of colloidal size paramagnetic particles was described by M. J. Davies, J. I. Taylor, N. Sachsinger, and I. J. Bruce (1998), Analytical Biochemistry 262: 92-94. The magnetic solid support was colloidal suspended magnetite particles (particle dimensions of 40 nm, 90 nm and 150 nm). Particles were added to a cell extract, and then a DNA precipitation agent, sodium iodide and isopropanol or sodium iodide and polyethylene glycol (PEG 8,000) were added, causing the DNA to flocculate out of solution phase and adsorb to particle surface. Work by Taylor et al., (2000), Journal of Chromatography A, 890: 159-166, compared DNA samples for FOR analysis isolated with plain magnetite colloidal particles or with magnetite silane polymer coating. Based on FOR, inhibitory substances seemed to be associated with DNA samples isolated with plain magnetite. The DNA samples isolated with silane-coated magnetite was not associated with FOR inhibition.

Another source of colloidal magnetite used for DNA purification is magnetosomes isolated from Magnetospiriliurn magneticum. The magnetite particles (50 nm to 100 nm across) can be coated with silaned polymer composed of 3-[2(2-aminoethyl)-ethylamino]propyl trimethoxysilane (AEEA-silane). This amine-rich silane coating acts as an affinity ligand, similar to diethylamine ethylene, for isolating DNA from cellular lysates. Like the silane coated magnetite particles described by Taylor et al., the surfaces of these AEEA-silane coated bacterial magnetite can be useful with DNA preparation for PCR analysis.

Paramagnetic mixed metal ferrite particles of cobalt ferrite were demonstrated to be useful solid supports for purifying DNA (see A. Spanova, B. Rittich, M. J. Benes, and D. Horak (2005), Journal of Chromatography A, 1080: 93 98). These ferrite particles were first coated with cross-linked alginate to improve DNA recovery.

Except for the magnetite isolated from bacteria, co-precipitation was the process used to synthesize the magnetite or the cobalt ferrite particles used in the works described previously. The co-precipitation process generally comprises adding a metal salt solution rich in iron salt, typically ferric salt, and an alkaline hydroxide solution, which induces the formation of ferrite particles.

As reported by W. C. Elmore (1938) Phys. Rev. 54; 309-310, one of the earliest descriptions of a co-precipitation method for synthesizing magnetite was described is by Le Fort, J. C. R. (1852) Acad. Sci. Paris, 34, 480. The La Fort method, which is the basis for most magnetite Formulations, is a metal salt solution composed of ferric salts and ferrous salts at molar ratios of 2 to 1, respectively, and the hydroxide solution is that of sodium hydroxide. Elmore's modification to the La Forte method was to treat the magnetite particles after synthesis with soap solution to coat the particles and prevent them from further oxidation to iron hydroxide.

The basic metal salt formula of the La Forte method is combination of trivalent or ferric iron at concentration twice the concentration of the divalent or ferrous iron. A number of variations for magnetite synthesis have been described and most are still based on La Forte method of 2 to 1 trivalent ion to divalent ion. Commonly cited examples of magnetite synthesis processes are in Massart, R. (1981) IEEE Transactions on Magnetics, MAG-17, 1247, the work of Phillips, A. P., van Bruggen, M. P. B., and Pathmamanovan, C. (1994) Langmuir 10, 92-99, and Matsuda, K., Sumida, M., Fufita, K., and Mitsuzawa, S., (1987) Bull, Chem. Soc. Japan. 60, 4441-4442. Common to all of these methods is the magnetite synthesis method of La Forte, the addition of hydroxide solution to iron salt solution composed of (trivalent) ferric salts and (divalent) ferrous salts in which the ferric salt is at a concentration that is greater than that of the ferrous salt.

The La Forte molar ratio of trivalent to divalent iron salts is the basis for synthesizing mixed metal ferrites, such as cobalt ferrites, nickel ferrites zinc ferrites, aluminum ferrites, gallium ferrites and boron ferrites. What is common to all of these formulations is that ferric salt is the source of iron for the ferrite particles. For many of these formulations, ferric salt is at the highest concentration. Examples of such methods are Wang et al., 2008, Journal of Alloys and Compounds 450: 276-283 (a method to synthesize cobalt ferrites), Iida at al., 2007, Journal of Colloid and Interface Science 314: 274-280 (describes two methods one for nickel ferrites and the other for zinc ferrites), and Wang et al., 2008, Journal of Alloys and Compounds 450: 276-283 (describes methods for making aluminum ferrite, boric ferrite, and gallium ferrite, in which for all three methods the source of iron as a ferric salt).

The work of I. H. Gul and A. Maqsood (2007), Journal of Magnetism and Magnetic Materials, 316: 13-18, describes a method for making mixed metal ferrite from metal salt solution which contained divalent cobalt nitrate, trivalent ferric nitrate, divalent zinc chloride, and tetravalent zirconium nitrate [Co(NO3)2, Fe(NO3)3, ZnCl2, and Zr(NO3)4] at molar ratios of 1 to 0.75 to 0.08 to 0.08, respectively. This formula differs from the other formulas based on the Le Forte formula in which the dominant salt is divalent, cobalt nitrate. However, like the above mixed metal ferrite formulas, the sole source of iron is a ferric salt.

The present invention features formulas for paramagnetic particles. The formulas of the present invention are based on metal salt solution formulas in which the sole source of iron is a ferrous salt. The formulas of the present invention comprise a tetravalent Group IV salt (metal salts that when oxidized via alkaline conditions do not produce paramagnetic particles). Examples of formulas of the present invention can be found in the figures. For all of the formulations, except for No. 1 (see FIG. 1), the concentration of the ferrous salt is greater than the Group IV salt. For the formulas disclosed, the molar ratio of ferrous iron to zirconium (formula No. 2-No. 40) ranges from 1 to 0.5 (formula No. 2) to 1 to 0.01 (formula No. 8).

For most of the formulations disclosed in this application, the dominant Group IV salt is zirconium (IV) chloride. In some embodiments, zirconium could be substituted with tetravalent titanium salt or hafnium salt in some of the three metal formulations. Without wishing to limit the present invention to any theory or mechanism, it is believed that particles made with the zirconium-free formulations of the present invention may have properties similar to the particles made with zirconium.

In some embodiments, the present invention features the substitution of traditional tetravalent zirconium salt with a blend of two or three tetravalent Group IV salts (e.g., the formulation comprises tetravalent zirconium salt, a tetravalent titanium salt, and/or a tetravalent hafnium salt).

In some embodiments, the present invention features the substitution of traditional tetravalent zirconium salt with a blend of the tetravalent salts of zirconium and titanium, zirconium and hafnium salts, and/or titanium and hafnium.

In nature, the mineral ilmenite is composed of 1 to 1 mix of ferrous oxide and tetravalent titanium oxide. This mineral is a common source for titanium. The magnetic responsiveness of this mineral compared to magnetite is considered in the art to be weak paramagnetic properties that would not be useful for bioprocessing based on magnetic separation. Since magnetic properties of ilmenite are considered to be weak, the formulations of the present invention (e.g., ferrous salts plus tetravalent Group IV salt) producing particles with strong paramagnetic properties were not obvious.

The ferrous-zirconium salt formulations of the present invention, which contain a third metal cation (other than titanium or hafnium), can be classified into three types: monovalent, divalent, and trivalent. In some embodiments, the monovalent formulations comprise lithium chloride at 0.1M (formula No. 9, see FIG. 1). In some embodiments, the divalent formulations comprise a Group 2 cation (e.g., barium chloride, calcium chloride, and magnesium chloride) at 0.1M (formula No. 10, 11, and 16). For the formulas with a different calcium content (e.g., formula No. 12 through 15) or a different magnesium content (e.g., formula No. 17 through 20), the properties of the particles were strongly paramagnetic and stable in water suspensions. DNA preparation exposed to these particles for prolonged periods of time did not acquire substances that inhibited PCR. A set of formulations based on barium salts (e.g., formula No. 31) with same range of molar ratios tested with calcium or magnesium salts may yield paramagnetic particles of the present invention, particles with properties similar to the particles produce from formula with calcium or magnesium salts.

Formulations with the divalent salt may comprise a divalent transition metal cation, e.g., cobalt chloride, nickel chloride, or zinc sulfate. Such particles had the properties of the particles of the invention, e.g., strongly paramagnetic, stable in water suspensions, and did not contaminate DNA samples so as to inhibit PCR. For two formulas that yielded particles of the present invention (No. 23 and No. 24), the concentration of cobalt ranged from 0.1 M to 0.03 molar. This that formulations No. 32, No. 33, and No. 34 (see FIG. 2) also yield particles of the present invention.

In some embodiments, the trivalent formulations comprise aluminum chloride or ferric (III) chloride (e.g., No. 21, No. 22). Formula No, 35 represents a formula based on trivalent aluminum salts. Formula No. 36 represents a formula comprising a gallium cation.

As shown in FIG. 2, in some embodiments, a set of trivalent transition metals may be used for formulations of the present invention. For example, the present invention features formulations ferric (III) chloride (e.g., No. 37) wherein the concentration of the ferric cation does not exceed a fifth of the concentration of the ferrous salt. The other trivalent metal cations for the present invention may include Group 5 metal cations (e.g., formula No. 38 based on vanadium chloride), Group 6 metal cations (e.g., formula No. 39 based on manganese chloride), and Group 7 metal cations (e.g., formula No. 40 based on chromium chloride).

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### DEFINITIONS

As used herein, terms common to chemistry and physics are based on definitions found in Gold Book, Compendium of Chemical Terminology, International Union of Pure and Applied Chemistry, Version 2.3.2, 2012-08-19. For example, the term colloidal refers to a state of subdivision, implying that the molecules or polymolecular particles dispersed in a medium have at least in one direction a dimension roughly between 1 nm and 1 μm, or that in a system, discontinuities are found at distances of that order. The term paramagnetic refers to substances having a magnetic susceptibility greater than 0. References to chemical elements and groupings associated with the Periodic Table refer to the group designations of the Jun. 1, 2012 version of the Periodic Table published by the International Union of Pure and Applied Chemistry (see http://www.iupac.org/fileadmin/user_upload/news/IUPAC_Periodic_Table-1Jun12.pdf).

### SUMMARY OF THE INVENTION

The present invention features paramagnetic particles synthesized by co-precipitation methods. The co-precipitation method may comprise mixing together an alkaline hydroxide solution (e.g., ammonium hydroxide, potassium hydroxide, sodium hydroxide, the like, or mixtures thereof) and a metal salt solution. The metal salt solutions are described in detail herein. The metal salt solution comprises at least one ferrous salt and at least one tetravalent metal salt selected from Group 4 elements of the Periodic Table. The concentration of the ferrous salt is equal to or greater than the concentration of the tetravalent metal salt.

In some embodiments, the tetravalent metal salt is at a concentration that is less than or equal to two-tenths the concentration of the ferrous salt. In some embodiments, the tetravalent metal salt is at a concentration that is less than or equal to one-tenth the concentration of the ferrous salt. In some embodiments, the tetravalent metal salt is at a concentration that is less than or equal to one-hundredth the concentration of the ferrous salt.

In some embodiments, the tetravalent metal salt comprises a zirconium salt. In some embodiments, the tetravalent salt comprises a titanium salt or a hafnium salt. In some embodiments, the tetravalent metal salt comprises a chloride salt, a sulfate salt, an acetate salt, an alkoxy salt, a halide salt, a nitrate salt, or a mixture thereof.

In some embodiments, the tetravalent salt is at a concentration that is one-tenth that of the ferrous salt, wherein the metal salt solution further comprises a secondary metal salt selected from Group 1, Group 2, Group 8, Group 9, Group 10, Group 12 or Group 13 elements of the Periodic Table. In some embodiments, the tetravalent salt is at a concentration that is less than or equal to one-tenth that of the ferrous salt, wherein the metal salt solution further comprises a secondary metal salt selected from Group 1, Group 2, Group 8, Group 9, Group 10, Group 12 or Group 13 elements of the Periodic Table. The secondary metal salt may be at a concentration less than that of the ferrous salt.

In some embodiments, the secondary metal salt comprises a lithium salt at a concentration between one-hundredth to two-tenths the concentration of the ferrous salt. In some embodiments, the secondary metal salt comprises a barium salt at a concentration between one-hundredth to two-tenths the concentration of the ferrous salt. In some embodiments, the secondary metal salt comprises a magnesium salt at a concentration between one-hundredth to two-tenths the concentration of the ferrous salt. In some embodiments, the secondary metal salt comprises a calcium salt at a concentration between one-hundredth to two-tenths the concentration of the ferrous salt. In some embodiments, the secondary metal salt comprises a ferric salt at a concentration between one-hundredth to two-tenths the concentration of the ferrous salt. In some embodiments, the secondary metal salt comprises a cobalt salt at a concentration that is between one-tenth to three-hundredth the concentration of the ferrous salt. In some embodiments, the secondary metal salt comprises a nickel salt at a concentration between one-hundredth to one-tenth the concentration of the ferrous salt. In some embodiments, the secondary metal salt comprises a zinc salt at a concentration between one-hundredth to one-tenth the concentration of the ferrous salt. In some embodiments, the secondary metal salt comprises an aluminum salt at a concentration between one-hundredth to one-tenth the concentration of the ferrous salt.

In some embodiments, the tetravalent salt is at a concentration that is one-tenth that of the ferrous salt, wherein the metal salt solution further comprises a secondary metal salts selected from Group 5, Group 6, or Group 7 elements of the Periodic Table. In some embodiments, the tetravalent salt is at a concentration that is less than or equal to one-tenth that of the ferrous salt, wherein the metal salt solution further comprises a secondary metal salts selected from Group 5, Group 6, or Group 7 elements of the Periodic Table.

In some embodiments, the secondary metal salt comprises vanadium salt at a concentration between one-hundredth to one-tenth the concentration of the ferrous salt. In some embodiments, the secondary metal salt comprises manganese salt at a concentration between one-hundredth to one-tenth the concentration of the ferrous salt. In some embodiments, the secondary metal salt comprises chromium salt at a concentration between one-hundredth to one-tenth the concentration of the ferrous salt.

In some embodiments, the paramagnetic particles are treated with a solution. The solution may, for example, comprise an anion, a glycol, a modified saccharide or derivative thereof, and/or a detergent or surfactant. In some embodiments, the anion is selected from the group consisting of: acetates, alkoxysilanes, borates, carbonates, carboxylates, citrates, fluoride, perchlorates, phosphates, phosphonates, sulfates, and sulfonates. In some embodiments, the glycol is selected from the group consisting of glycerol, ethylene glycol, 1,2 propylene glycol, 1,3 propylene glycol, glycerol, 1,2 butylene glycol, 1,3 butylene glycol, 2,3 butylene glycol, polyglycol polymers including polyethylene glycol and polypropylene glycol. In some embodiments, the modified saccharide or derivative thereof is selected from the group consisting of mannitol, sorbitol, or erythritol. In some embodiments, the detergent or surfactant is selected from the group consisting of dodecyl sulfates, lauroyl sarcosines, polyoxyethylenesorbitans, oleic acid, palmatic acid, octanoic acid, sulfate detergents, phosphate detergents, and carbonate detergents.

In some embodiments, a surface of the paramagnetic particles is coated with silane polymer comprising monomers selected from ethyltriethoxysilane, 3-triethoxysilylpropylamine, 3-(trimethoxysilyl)-1-propanethial, 3-(2,3-epoxypropyloxy) propyltriethoxysilane, or mixtures thereof. In some embodiments, the paramagnetic particles function to bind biomolecules by covalent linkage of divinyl sulfone, aldhahydes, or succinamides to amines, epoxy, or thio substituted silanes linked to a particle surface.

The present invention also features kits comprising paramagnetic particles of the present invention.

The present invention also features paramagnetic particles according to the present invention used in methods. Methods may comprise separating, isolating, purifying, fractionating, concentrating, or detecting a biomolecule. The biomolecule may comprise (but is not limited to) nucleic acids, oligonucleotides, proteins, polypeptides, peptides, carbohydrates, lipids, or combinations thereof.

The present invention also features methods for purifying or isolating biomolecules, e.g., a nucleic acid, from a biological source. In some embodiments, the method comprises (a) disrupting of the biological source and dissolution of the nucleic acid using an extraction buffer comprising at least a detergent, one chaotropic agent, and a protease enzyme; (b) adding a set of paramagnetic particles according to the present invention to the mixture of step (a) and mixing until the set of paramagnetic particles are evenly distributed throughout the solution; (c) adding a nucleic acid precipitating agent to the mixture of step (b) to a sufficient quantity so as to induce the nucleic acid to adsorb to a surface of the paramagnetic particles; (d) concentrating the paramagnetic particles out from suspension phase to a pellet by the use of a magnetic field; (e) removing a liquid phase from the pellet of paramagnetic particles; (f) suspending the paramagnetic particles in an elution solution which causes the nucleic acid to partition back to solution phase; and (g) removing a paramagnetic metal oxide particle from suspension by use of a magnetic field and retrieving a remaining nucleic acid solution by transfer to a new vessel.

In some embodiments, the nucleic acid extraction buffer comprises at least one of (a) a chaotropic compound selected from guanidine salts, urea, formamide, or isothiocyanate salts; (b) a detergent selected from sodium dodecyl sulfate, N Lauroylsacosine, sodium [dodecanoyl (methyl) amino]acetate, hexadecyl-trimethyl-ammonium bromide, t-octylphenoxypolyethoxyethanol, polyoxyethylene (20) sorbitan monooleate, polyoxyethylene (20) sorbitan monolaurate; (c) a protease enzyme selected from proteinase K, protease substilisin, and serine type proteases; (d) nucleic acid precipitating agent selected from ethanol, 2-propanol, polyethylene glycol, guanidine salts, sodium iodide, sodium perchlorate, lithium chloride, hexadecyltrimethylammonium bromide, spermine, spermidine, or mixtures thereof.

### BRIEF DESCRIPTION OF THE DRAWINGS

FIG. 1 is a chart illustrating various metal salt formulations for particle synthesis. The formulations in FIG. 1 are based on solutions comprising ammonium hydroxide as the hydroxide solution.

FIG. 2 is a chart illustrating examples of formulations for particle synthesis. The formulas were derived from formula No. 5 comprising ferrous (II) chloride at 1 M and zirconium (IV) chloride at 0.1 M.

### DESCRIPTION OF PREFERRED EMBODIMENTS

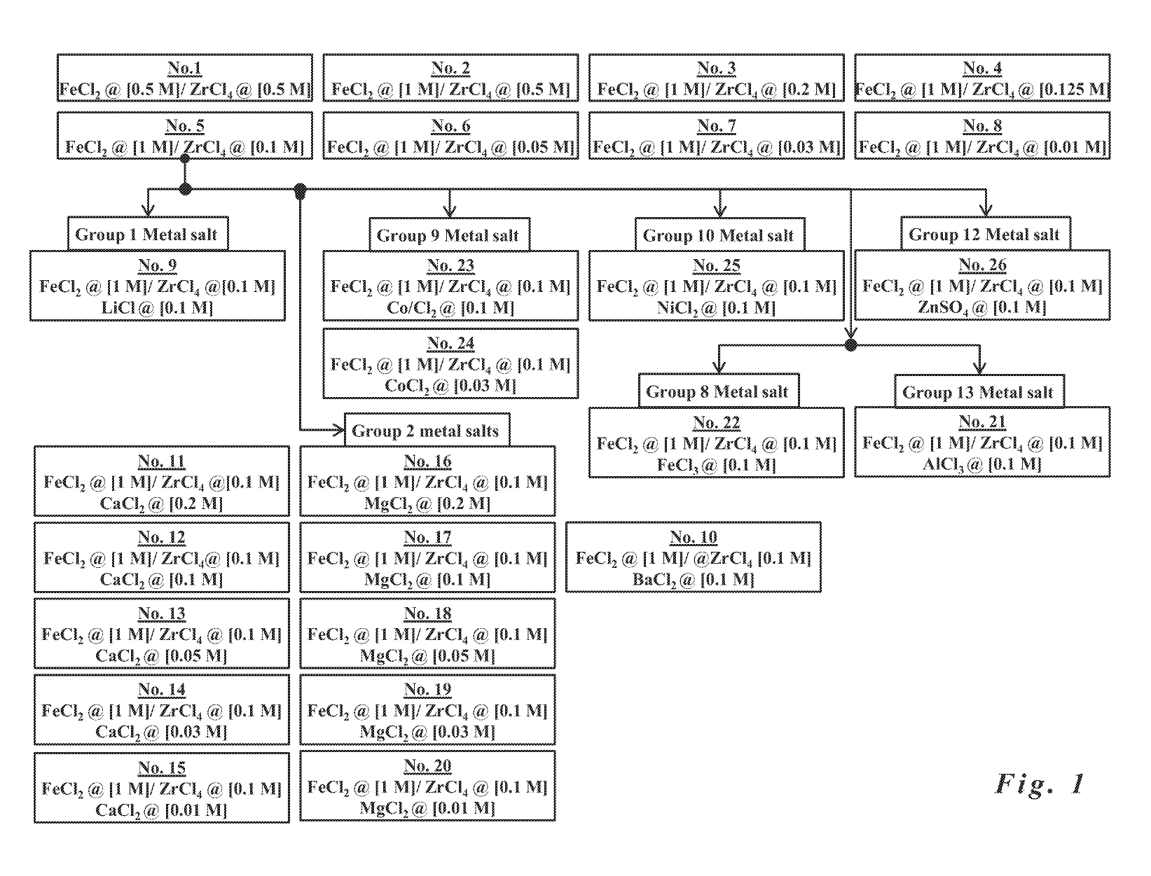

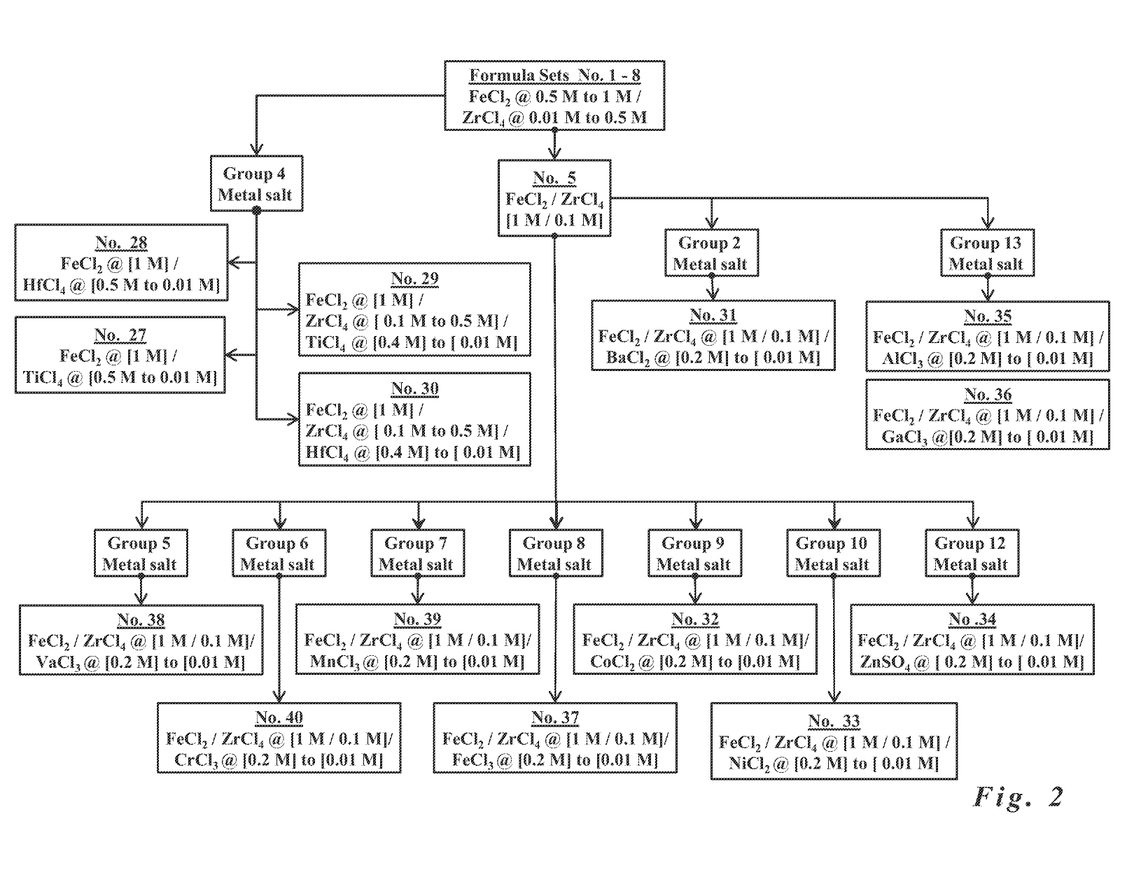

Referring now to FIG. 1-2, the present invention features formulations for the synthesis of paramagnetic particles and methods utilizing such particles (e.g., for biochemical applications, e.g., FOR). For example, the present invention features paramagnetic particles synthesized from a metal salt solution comprising a ferrous salt and a tetravalent Group 4 metal salt. Examples of such formulations are displayed in FIG. 1 and FIG. 2 (e.g., No. 1 through No. 8 for formulations composed solely of the two metal cations). The molar ratio of ferrous (II) chloride and zirconium (IV) chloride can range from equal molar, for zirconium formulations, to a ratio in which the ferrous salt is 100 fold greater than the zirconium (IV) chloride. For the majority of the formulations disclosed or anticipated in the Figures, the molar ratio of ferrous salt to zirconium salt is 10 to 1.

For most examples, metal chloride salts were used except for formulations with zinc sulfate. Ferrite particles may be made with metal salts of other halides, acetate, sulfate, nitrate, and alkoxy anions. In some embodiments, the hydroxide solution used to synthesize particles of the present invention comprises concentrated ammonium hydroxide. However, the present invention is not limited to concentrated ammonium hydroxide. For example, in some embodiments, the hydroxide solution comprises sodium, potassium, cesium or mixtures thereof.

In some embodiments, the co-precipitation processes used to synthesize the paramagnetic particles of the invention are performed under ambient conditions. However, the present invention is not limited to ambient conditions; for example, in some embodiments, co-precipitation is performed at temperatures and pressures that are higher than ambient conditions. In some embodiments, the co-precipitation method can be accelerated with the application of microwave radiation.

Particles synthesized via formulations of the present invention may be used for a variety of applications. Examples of applications include but are not limited to DNA purification, immunoassays, protein precipitation, nucleic acid hybridization, and/or DNA amplification. As an example, in some embodiments, particles are used in a hybridization-based assay such as a microarray, wherein particles are coated with DNA probes. In some embodiments, particles are used in an immunoassay wherein the particles are bound with a specific antibody or with a specific antigen.

In some embodiments, particles of the present invention are mixed with larger micron size paramagnetic beads to produce a blended solid phase having the magnetic mass attributed to the micron size beads mixed with the larger surface area. The micron particle-mixed media may be an effective means to increase the sensitivity and speed for most magnetic bead based assays.

In some embodiments, particles of the invention may be incorporated in kits for purifying nucleic acids, RNA or DNA, for the detection of biomolecules, and/or for diagnosis of disease. In some embodiments, particles of the invention may be incorporated in kits designed for automated processing.

The present invention is directed to formulations of metal salt solutions used to synthesize paramagnetic particles, e.g., by co-precipitation methods initiated by the ammonium hydroxide. These formulations comprise at least two metal salts: a ferrous salt and at least one tetravalent salt of Group 4 (of the Periodic Table). In some embodiments, the concentration of the ferrous salt is the same or greater than the concentration of the Group 4 metal salt. In some embodiments, the tetravalent zirconium salt is zirconium (IV) chloride and at a concentration that is 10 fold less than the concentration of the ferrous salt.

The metal salt formulations of the invention can be classified into two classes, metal solutions composed solely of ferrous salt and tetravalent Group 4 metal salt. The second class of formulations contain three metal salts, ferrous salt, Group 4 metal salt, and a third metal salt selected the elements of the Groups 1 and 2. Groups 5 through 10, and Groups 12 and 13 of the Periodic Table.

In some embodiments, in the three metal salt formulations, the concentration of the zirconium (IV) chloride and the third metal salt does not exceed the concentration of the ferrous salt, e.g., for the zirconium (IV) chloride concentrations that ranged from equal to one hundred fold less than the concentration of the ferrous (II) chloride. Examples of the three metals cation formulations may comprise ferrous (II) chloride and zirconium (IV) chloride at molar ratio of 10 to 1 with the concentration of third metal salt ranged is 5 fold to 100 fold less than ferrous (II) chloride. In some embodiments, a set of metal salt solutions applicable for the invention may include those based on metal salts of halide salts, nitrate salts, sulfate salts, acetate salts, alkoxy salts, or mixtures thereof.

The present invention also features formulations with the metal salt solutions wherein the Group 4 metal salt is tetravalent titanium salt or a tetravalent hafnium salt. The forms of these tetravalent metal salts when mixed with alcohols form tetra alkoxy metal complexes, which could be considered a salt. For example, a reaction of tetravalent halide salt of titanium with isopropyl alcohol forms tetra isopropyl orthotitanates. Other alcohols may include methanol, ethanol, propyl alcohols, or the butanols or mixtures thereof.

The present invention features paramagnetic particles synthesized from various formulations described herein. In some embodiments, the formula comprises a two metal salt solution comprising 0.5 M ferrous (II) chloride and 0.5 M zirconium (IV) chloride (e.g., formula No. 1, see FIG. 1). In some embodiments, the formula comprises a two metal salt solution comprising 1.0 M ferrous (II) chloride and 0.5 M zirconium (IV) chloride (e.g., formula No. 2, see FIG. 1). In some embodiments, the formula comprises a two metal salt solution comprising 1.0 M ferrous (II) chloride and 0.2 M zirconium (IV) chloride (e.g., formula No. 3, see FIG. 1). In some embodiments, the formula comprises a two metal salt solution comprising 1.0 M ferrous (II) chloride and 0.125 M zirconium (IV) chloride (e.g., formula No. 4, see FIG. 1). In some embodiments, the formula comprises a two metal salt solution comprising 1.0 M ferrous (II) chloride and 0.1 M zirconium (IV) chloride (e.g., formula No. 5, see FIG. 1). In some embodiments, the formula comprises a two metal salt solution comprising 1.0 M ferrous (II) chloride and 0.05 M zirconium (IV) chloride (e.g., formula No. 6, see FIG. 1). In some embodiments, the formula comprises a two metal salt solution comprising 1.0 M ferrous (II) chloride and 0.03 M zirconium (IV) chloride (e.g., formula No. 7, see FIG. 1). In some embodiments, the formula comprises a two metal salt solution comprising 1.0 M ferrous (II) chloride and 0.01 M zirconium (IV) chloride (e.g., formula No. 8, see FIG. 1).

In some embodiments, the formula comprises a three metal salt solution comprising 1.0 M ferrous (II) chloride and 0.1 M zirconium (IV) chloride, and titanium tetrachloride at a concentration that can range from 0.01 M to 0.4 M (e.g., formula No. 29, see FIG. 2). In some embodiments, the formula comprises a three metal salt solution comprising 1.0 M ferrous (II) chloride and 0.1 M zirconium (IV) chloride, and hafnium tetrachloride at a concentration that can range from 0.01 M to 0.4 M (e.g., formula No. 30, see FIG. 2). In some embodiments, the formula comprises a three metal salt solution comprising 1.0 M ferrous (II) chloride and 0.1 M zirconium (IV) chloride, and 0.1 M lithium chloride (e.g., formula No. 9, see FIG. 1).

In some embodiments, the formula comprises a three metal salt solution comprising 1.0 M ferrous (II) chloride and 0.1 M zirconium (IV) chloride, and 0.1 M barium (II) chloride (e.g., formula No. 10, see FIG. 1). In some embodiments, the formula comprises a three metal salt solution comprising 1.0 M ferrous (II) chloride and 0.1 M zirconium (IV) chloride, and barium (II) chloride at a concentration that can range from 0.01 M to 0.2 M (e.g., formula No. 31, see FIG. 2). In some embodiments, the formula comprises a three metal salt solution comprising 1.0 M ferrous (II) chloride and 0.1 M zirconium (IV) chloride, and 0.1 M calcium (II) chloride (e.g., formula No. 11, see FIG. 1).

In some embodiments, the formula comprises a three metal salt solution comprising 1.0 M ferrous (II) chloride and 0.1 M zirconium (IV) chloride, and 0.2 M calcium (II) chloride (e.g., formula No. 12, see FIG. 1). In some embodiments, the formula comprises a three metal salt solution comprising 1.0 M ferrous (II) chloride and 0.1 M zirconium (IV) chloride, and 0.05 M calcium (II) chloride (e.g., formula No. 13, see FIG. 1). In some embodiments, the formula comprises a three metal salt solution comprising 1.0 M ferrous (H) chloride and 0.1 M zirconium (IV) chloride, and 0.03 M calcium (H) chloride (e.g., formula No, 14, see FIG. 1). In some embodiments, the formula comprises a three metal salt solution comprising 1.0 M ferrous (II) chloride and 0.1 M zirconium (IV) chloride, and 0.01 M calcium (H) chloride (e.g., formula No. 15, see FIG. 1).

In some embodiments, the formula comprises a three metal salt solution comprising 1.0 M ferrous (H) chloride and 0.1 M zirconium (IV) chloride, and 0.1 M magnesium (H) chloride (e.g., formula No. 16, see FIG. 1). In some embodiments, the formula comprises a three metal salt solution comprising 1.0 M ferrous (II) chloride and 0.1 M zirconium (IV) chloride, and 0.2 M magnesium (H) chloride (e.g., formula No. 17, see FIG. 1). In some embodiments, the formula comprises a three metal salt solution comprising 1.0 M ferrous (II) chloride and 0.1 M zirconium (IV) chloride, and 0.05 M magnesium (H) chloride (e.g., formula No. 18, see FIG. 1). In some embodiments, the formula comprises a three metal salt solution comprising 1.0 M ferrous (H) chloride and 0.1 M zirconium (IV) chloride, and 0.03 M magnesium (II) chloride (e.g., formula No. 19, see FIG. 1). In some embodiments, the formula comprises a three metal salt solution comprising 1.0 M ferrous (II) chloride and 0.1 M zirconium (IV) chloride, and 0.01 M magnesium (II) chloride (e.g., formula No. 20, see FIG. 1). In some embodiments, the formula comprises a three metal salt solution comprising 1.0 M ferrous (II) chloride and 0.1 M zirconium (IV) chloride, and 0.1 M aluminum (III) chloride (e.g., formula No. 21, see FIG. 1).

In some embodiments, the formula comprises a three metal salt solution comprising 1.0 M ferrous (II) chloride and 0.1 M zirconium (IV) chloride, and aluminum (III) chloride at a concentration that can range from 0.01 M to 0.2 M (e.g., formula No. 35, see FIG. 2). In some embodiments, the formula comprises a three metal salt solution comprising 1.0 M ferrous (II) chloride and 0.1 M zirconium (IV) chloride, and gallium (HI) chloride at a concentration that can range from 0.01 M to 0.2 M (e.g., formula No. 36, see FIG. 2), In some embodiments, the formula comprises a three metal salt solution comprising 1.0 M ferrous (II) chloride and 0.1 M zirconium (IV) chloride, and 0.1 M ferric (III) chloride (e.g., formula No. 22, see FIG. 1). In some embodiments, the formula comprises a three metal salt solution comprising 1.0 M ferrous (II) chloride and 0.1 M zirconium (IV) chloride, and ferric (III) chloride at a concentration that can range from 0.01 M to 0.2 M (e.g., formula No. 37, see FIG. 2).

In some embodiments, the formula comprises a three metal salt solution comprising 1.0 M ferrous (II) chloride and 0.1 M zirconium (IV) chloride, and 0.1 M cobalt (II) chloride (e.g., formula No. 23, see FIG. 1). In some embodiments, the formula comprises a three metal salt solution comprising 1.0 M ferrous (II) chloride and 0.1 M zirconium (IV) chloride, and 0.03 M cobalt (II) chloride (e.g., formula No. 24, see FIG. 1). In some embodiments, the formula comprises a three metal salt solution comprising 1.0 M ferrous (II) chloride and 0.1 M zirconium (IV) chloride, and cobalt (II) chloride at a concentration that can range from 0.01 M to 0.2 M (e.g., formula No. 32, see FIG. 2). In some embodiments, the formula comprises a three metal salt solution comprising 1.0 M ferrous (II) chloride and 0.1 M zirconium (IV) chloride, and 0.1 M nickel (II) chloride (e.g., formula No. 25, see FIG. 1).

In some embodiments, the formula comprises a three metal salt solution comprising 1.0 M ferrous (II) chloride and 0.1 M zirconium (IV) chloride, and nickel (II) chloride at a concentration that can range from 0.01 M to 0.2 M (e.g., formula No. 33, see FIG. 2).

In some embodiments, the formula comprises a three metal salt solution comprising 1.0 M ferrous (II) chloride and 0.1 M zirconium (IV) chloride, and 0.1 M zinc (II) sulfate (e.g., formula No. 26, see FIG. 1). In some embodiments, the formula comprises a three metal salt solution comprising 1.0 M ferrous (II) chloride and 0.1 M zirconium (IV) chloride, and zinc (II) sulfate at a concentration that can range from 0.01 M to 0.2 M (e.g., formula No. 34, see FIG. 2). In some embodiments, the formula comprises a three metal salt solution comprising 1.0 M ferrous (II) chloride and 0.1 M zirconium (IV) chloride, and vanadium (III) chloride at a concentration that can range from 0.01 M to 0.2 M (e.g., formula No. 38, see FIG. 2). In some embodiments, the formula comprises a three metal salt solution comprising 1.0 M ferrous (II) chloride and 0.1 M zirconium (IV) chloride, and manganese (III) chloride at a concentration that can range from 0.01 M to 0.2 M (e.g., formula No. 39, see FIG. 2). In some embodiments, the formula comprises a three metal salt solution comprising 1.0 M ferrous (II) chloride and 0.1 M zirconium (IV) chloride, and chromium (III) chloride at a concentration that can range from 0.01 M to 0.2 M (e.g., formula No. 40, see FIG. 2).

The present invention also features particles synthesized from solutions of metal salts comprised of ferrous salts and tetravalent Group 4 metal salts that were further treated with carbon polymers, silicon polymers, or polymers of biological origin as a coating or embedded within beads for the purpose of biological applications.

The present invention also features particles synthesized from solutions of metal salts comprised of ferrous salts and tetravalent Group 4 metal salts that were further treated with anions or mixtures thereof selected from but not limited to phosphates, sulfates, carbonates, borates, fluoride, carboxylates, phosphonates, or sulfonates.

The present invention also features particles synthesized from solutions of metal salts comprised of ferrous salts and tetravalent Group 4 metal salts that were further treated with a glycol or mixtures thereof selected from but not limited to ethylene glycol, 1,2 propylene glycol, 1,3 propylene glycol, glycerol, 1,2 butylene glycol, 1,3 butylene glycol, 2,3 butylene glycol, glycol polymers including polyethylene glycol and polypropylene glycol, alcohol derivatives of hexoses including mannitol, and sorbitol.

The present invention also features particles synthesized from solutions of metal salts comprised of ferrous salts and tetravalent Group 4 metal salts that were further treated with detergents or surfactants or mixtures thereof selected from but not limited to detergents based on dodecyl sulfate, lauroyl sarcosine, oleic acid, palmatic acid, octanoic acid, polyoxyethylenesorbitans, or the class of detergents which is a phosphate derivative.

The present invention also features particles synthesized from solutions of metal salts comprised of ferrous salts and tetravalent Group 4 metal salts of colloidal dimensions and treated with anions, glycols, and detergents or surfactants as single solutions or mixtures, for the solid-phase processes directed toward isolating, purifying, concentrating, or the detection of nucleic acids from biological sources, included in methods for their use, and kits for performing these processes, in which the sources for these molecules of interest include cells, tissues or bodily fluids, semi-purified preparations or dilute solutions, including methods for their use, and kits comprising these treated metal oxide particles for performing these methods.

The present invention also features particles synthesized from solutions of metal salts comprised of ferrous salts and tetravalent Group 4 metal salts of colloidal dimensions and treated with anions, glycols, and detergents suspended at concentration ranging from 0.2 to 40 mg/mL in a solution comprised of Na2HPO4, Na3PO4, H3BO3, Na2B4O7, NaF, with propylene glycol or polyethylene glycol (˜8,000 MW), Triton X-100.

The present invention also features particles synthesized from solutions of metal salts comprised of ferrous salts and tetravalent Group 4 metal salts of colloidal dimensions, wherein the particles are used as the solid phase for purifying, isolating, or concentrating nucleic acids.

In some embodiments, the particles of the present invention are used in methods. Methods may include but are not limited to (a) extraction of biological samples with buffers that contain chaotropic molecules, detergents, and proteases; (b) methods that utilize nucleic acid precipitation agents to cause nucleic acids to adsorb to the suspended particles, precipitation agents that are selected from ethyl or propyl alcohols, polyglycols, guanidine, spermidine, lithium salts or iodide salts or mixtures thereof; (c) methods that partition DNA adsorbed to the surface of the particles by use of magnetic field; (d) processes that elute or dissolve adsorbed DNA or RNA from the particle surface by suspending the particles buffer agents such as tris(hydroxymethyl)aminomethane, ethylenediaminetetraacetic acid, 3-(N-morpholino)propanesulfonic acid, borate, phosphates, or chloride salts, or mixtures thereof, at pH which can range from pH 6 to pH 11, and at ionic concentration and at temperatures that support the dissolution of the DNA or RNA from the particles surface.

In some embodiments, the paramagnetic particles of the present invention are used as solid support to enrich, isolate, purify or concentrate proteins by the methods based on protein flocculation or precipitation agents known in the art, including alcohols, glycol polymers such as polyethylene glycol, acetate salts, lithium salts sulfate salts, caproic acid, or mixtures thereof, which when mixed at appropriate concentrations induce the targeted protein or proteins to aggregate with and adsorb to the paramagnetic particles in suspension phase. The proteins adsorbed or aggregated with paramagnetic particles are partitioned from suspension phase by used of magnetic field, which concentrates the particles out of suspension. Once partitioned to a pellet, the supernatant fraction is removed, and the paramagnetic particle-protein complexes dispersed in a solution that elutes the proteins from the paramagnetic particles.

In some embodiments, the particles of the present invention are used in methods that utilize buffers containing buffering agents such as tris(hydroxymethyl) aminomethane, ethylenediamine tetraacetic acid, 3-(N-morpholino) propane sulfonic acid, borate, of phosphates, or of chloride, or mixtures thereof, at pH range, ionic concentration, and at temperatures that would allow the protein to dissolve back to solution phase without denaturing the protein.

### Examples

The following examples describe various embodiments of the present invention. The present invention is not limited to the examples of formulations and methods described herein.

### Example 1

Paramagnetic particles synthesized by adding concentrated NH4OH solution to a solution composed of 1 M ferrous (II) chloride and 0.1 M zirconium (IV) chloride in HCl buffer, pH 1, to a final concentration of about 10%. Added to this mixture is 8.6 volumes of water and the final mixture was incubated for 20 hours at ambient temperature and pressure. After this incubation, paramagnetic particles were concentrated to a pellet by magnetic field. After particles are collected the remaining supernatant was removed from the particle pellet and discarded. After the supernatant was removed, particles were subjected to a series of solution exchanges consisting of dispersing the particles in suspension phase and collecting the particles by use of magnetic field. The volume of each set of the dispersion-pelleting process was same as the volume of particle suspension when incubated for 20 hours in solution containing ammonium hydroxide. The solutions used for the dispersion-pelleting process are listed in the order of use: deionized water, repeated once, 100 mM Na2CO3 incubated for 24 hours, and 100 mM Na2CO3. The particle pellet is dispersed and stored in 0.6 volumes of 100 mM Na2CO3.

### Example 2

Paramagnetic particles of the invention synthesized by co-precipitation method of Example 1 in which the metal salt solution composed of 0.5 M ferrous (H) chloride and 0.5 M zirconium (IV) chloride.

### Example 3

Paramagnetic particles of the invention synthesized by co-precipitation method of Example 1 in which the metal salt solution composed of 1.0 M ferrous (II) chloride and 0.5 M zirconium (IV) chloride.

### Example 4

Paramagnetic particles of the invention synthesized by co-precipitation method of Example 1 in which the metal salt solution composed of 1.0 M ferrous (II) chloride and 0.2 M zirconium (IV) chloride.

### Example 5

Paramagnetic particles of the invention synthesized by co-precipitation method of Example 1 in which the metal salt solution composed of 1.0 M ferrous (II) chloride and 0.125 M zirconium (IV) chloride.

### Example 6

Paramagnetic particles of the invention synthesized by co-precipitation method of Example 1 in which the metal salt solution composed of 1.0 M ferrous (H) chloride and 0.1 M zirconium (IV) chloride.

### Example 7

Paramagnetic particles of the invention synthesized by co-precipitation method of Example 1 in which the metal salt solution composed of 1.0 M ferrous (H) chloride and 0.05 M zirconium (IV) chloride.

### Example 8

Paramagnetic particles of the invention synthesized by co-precipitation method of Example 1 in which the metal salt solution composed of 1.0 M ferrous (II) chloride and 0.03 M zirconium (IV) chloride.

### Example 9

Paramagnetic particles of the invention synthesized by co-precipitation method of Example 1 in which the metal salt solution composed of 1.0 M ferrous (II) chloride and 0.01 M zirconium (IV) chloride.

### Example 10

Paramagnetic particles of the invention synthesized by co-precipitation method of Example 1 in which the metal salt solution composed of 1.0 M ferrous (H) chloride, 0.1 M zirconium (IV) chloride, and 0.1 M lithium chloride.

### Example 11

Paramagnetic particles of the invention synthesized by co-precipitation method of Example 1 in which the metal salt solution composed of 1.0 M ferrous (H) chloride. 0.1 M zirconium (IV) chloride, and 0.1 M barium chloride.

### Example 12

Paramagnetic particles of the invention synthesized by co-precipitation method of Example 1 in which the metal salt solution composed of 1.0 M ferrous (H) chloride, 0.1 M zirconium (IV) chloride, and 0.2 M calcium chloride.

### Example 13

Paramagnetic particles of the invention synthesized by co-precipitation method of Example 1 in which the metal salt solution composed of 1.0 M ferrous (II) chloride, 0.1 M zirconium (IV) chloride, and 0.1 M calcium chloride.

### Example 14

Paramagnetic particles of the invention synthesized by co-precipitation method of Example 1 in which the metal salt solution composed of 1.0 M ferrous (H) chloride, 0.1 M zirconium (IV) chloride, and 0.05 M calcium chloride.

### Example 15

Paramagnetic particles of the invention synthesized by co-precipitation method of Example 1 in which the metal salt solution composed of 1.0 M ferrous (II) chloride, 0.1 M zirconium (IV) chloride, and 0.03 M calcium chloride.

### Example 16

Paramagnetic particles of the invention synthesized by co-precipitation method of Example 1 in which the metal salt solution composed of 1.0 M Ferrous (II) chloride, 0.1 M zirconium (IV) chloride, and 0.01 M calcium chloride.

### Example 17

Paramagnetic particles of the invention synthesized by co-precipitation method of Example 1 in which the metal salt solution composed of 1.0 M ferrous (II) chloride, 0.1 M zirconium (IV) chloride, and 0.2 M magnesium chloride.

### Example 18

Paramagnetic particles of the invention synthesized by co-precipitation method of Example 1 in which the metal salt solution composed of 1.0 M ferrous (II) chloride, 0.1 M zirconium (IV) chloride, and 0.1 M magnesium chloride.

### Example 19

Paramagnetic particles of the invention synthesized by co-precipitation method of Example 1 in which the metal salt solution composed of 1.0 M ferrous (H) chloride, 0.1 M zirconium (IV) chloride, and 0.05 M magnesium chloride.

### Example 20

Paramagnetic particles of the invention synthesized by co-precipitation method of Example 1 in which the metal salt solution composed of 1.0 M ferrous (H) chloride, 0.1 M zirconium (IV) chloride, and 0.03 M magnesium chloride.

### Example 21

Paramagnetic particles of the invention synthesized by co-precipitation method of Example 1 in which the metal salt solution composed of 1.0 M ferrous (II) chloride, 0.1 M zirconium (IV) chloride, and 0.01 M magnesium chloride.

### Example 22

Paramagnetic particles of the invention synthesized by co-precipitation method of Example 1 in which the metal salt solution composed of 1.0 M ferrous (II) chloride, 0.1 M zirconium (IV) chloride, and 0.1 M aluminum (III) chloride.

### Example 23

Paramagnetic particles of the invention synthesized by co-precipitation method of Example 1 in which the metal salt solution composed of 1.0 M ferrous (II) chloride, 0.1 M zirconium (IV) chloride, and 0.1 M ferric (III) chloride.

### Example 24

Paramagnetic particles of the invention synthesized by co-precipitation method of Example 1 in which the metal salt solution composed of 1.0 M ferrous (II) chloride, 0.1 M zirconium (IV) chloride, and 0.1 M cobalt (II) chloride.

### Example 25

Paramagnetic particles of the invention synthesized by co-precipitation method of Example 1 in which the metal salt solution composed of 1.0 M ferrous (II) chloride, 0.1 M zirconium (IV) chloride, and 0.03 M cobalt (II) chloride.

### Example 26

Paramagnetic particles of the invention synthesized by co-precipitation method of Example 1 in which the metal salt solution composed of 1.0 M ferrous (II) chloride, 0.1 M zirconium (IV) chloride, and 0.1 M nickel (II) chloride.

### Example 27

Paramagnetic particles of the invention synthesized by co-precipitation method of Example 1 in which the metal salt solution composed of 1.0 M ferrous (H) chloride, 0.1 M zirconium (IV) chloride, and 0.1 M zinc (II) sulfate.

### Example 28

Paramagnetic particles of the invention which could be synthesized by co-precipitation method of Example 1 from a set of metal salt solutions composed of 1.0 M ferrous (H) chloride, 0.1 M zirconium (IV) chloride, and lithium chloride at a concentration that can range from 0.2 M to 0.01 M.

### Example 28A

Paramagnetic particles of the invention which could be synthesized by co-precipitation method of Example 1 from a set of metal salt solutions composed of 1.0 M ferrous (II) chloride, 0.1 M zirconium (IV) chloride, and barium chloride at a concentration that can range from 0.2 M to 0.01 M.

### Example 29

Paramagnetic particles of the invention which could be synthesized by co-precipitation method of Example 1 from a set of metal salt solutions composed of 1.0 M ferrous (II) chloride, 0.1 M zirconium (IV) chloride, and cobalt (II) chloride at a concentration that can range from 0.2 M to 0.01 M.

### Example 29A

Paramagnetic particles of the invention which could be synthesized by co-precipitation method of Example 1 from a set of metal salt solutions composed of 1.0 M ferrous (II) chloride, 0.1 M zirconium (IV) chloride, and zinc (II) chloride at a concentration that can range from 0.2 M to 0.01 M.

### Example 29B

Paramagnetic particles of the invention which could be synthesized by co-precipitation method of Example 1 from a set of metal salt solutions composed of 1.0 M ferrous (II) chloride, 0.1 M zirconium (IV) chloride, and manganese (H) chloride at a concentration that can range from 0.2 M to 0.01 M.

### Example 29C

Paramagnetic particles of the invention which could be synthesized by co-precipitation method of Example 1 from a set of metal salt solutions composed of 1.0 M ferrous (II) chloride, 0.1 M zirconium (IV) chloride, and nickel (II) chloride at a concentration that can range from 0.2 M to 0.01 M.

### Example 29D

Paramagnetic particles of the invention which could be synthesized by co-precipitation method of Example 1 from a set of metal salt solutions composed of 1.0 M ferrous (II) chloride, 0.1 M zirconium (IV) chloride, and ferric (III) chloride at a concentration that can range from 0.2 M to 0.01 M.

### Example 29E

Paramagnetic particles of the invention which could be synthesized by co-precipitation method of Example 1 from a set of metal salt solutions composed of 1.0 M ferrous (II) chloride, 0.1 M zirconium (IV) chloride, and chromium (III) chloride at a concentration that can range from 0.2 M to 0.01 M.

### Example 29F

Paramagnetic particles of the invention which could be synthesized by co-precipitation method of Example 1 from a set of metal salt solutions composed of 1.0 M ferrous (II) chloride, 0.1 M zirconium (IV) chloride, and aluminum (III) chloride at a concentration that can range from 0.2 M to 0.01 M.

### Example 29G

Paramagnetic particles of the invention which could be synthesized by co-precipitation method of Example 1 from a set of metal salt solutions composed of 1.0 M ferrous (II) chloride, 0.1 M zirconium (IV) chloride, and vanadium (III) chloride at a concentration that can range from 0.2 M to 0.01 M.

### Example 29H

Paramagnetic particles of the invention which could be synthesized by co-precipitation method of Example 1 from a set of metal salt solutions composed of 0.5 M ferrous (II) chloride, 0.1 M zirconium (IV) chloride, and titanium (IV) chloride at a concentration that can range from 0.4 M to 0.01 M.

### Example 29I

Paramagnetic particles of the invention which could be synthesized by co-precipitation method of Example 1 from a set of metal salt solutions composed of 0.5 M ferrous (II) chloride, 0.1 M zirconium (IV) chloride, and hafnium (IV) chloride at a concentration that can range from 0.4 M to 0.01 M.

### Example 37

Paramagnetic particles of the invention which could be synthesized by co-precipitation method of Example 1 in which the metal salt solution composed of 0.5 M ferrous (II) chloride and 0.5 M titanium tetrachloride.

### Example 38

Paramagnetic particles of the invention which could be synthesized by co-precipitation method of Example 1 in which the metal salt solution composed of 1.0 M ferrous (H) chloride and 0.5 M titanium tetrachloride.

### Example 39

Paramagnetic particles of the invention which could be synthesized by co-precipitation method of Example 1 in which the metal salt solution composed of 1.0 M ferrous (H) chloride and 0.2 M titanium tetrachloride.

### Example 40

Paramagnetic particles of the invention which could be synthesized by co-precipitation method of Example 1 in which the metal salt solution composed of 1.0 M ferrous (H) chloride and 0.125 M titanium tetrachloride.

### Example 41

Paramagnetic particles of the invention which could be synthesized by co-precipitation method of Example 1 in which the metal salt solution composed of 1.0 M ferrous (II) chloride and 0.1 M titanium tetrachloride.

### Example 42

Paramagnetic particles of the invention which could be synthesized by co-precipitation method of Example 1 in which the metal salt solution composed of 1.0 M ferrous (II) chloride and 0.05 M titanium tetrachloride.

### Example 43

Paramagnetic particles of the invention which could be synthesized by co-precipitation method of Example 1 in which the metal salt solution composed of 1.0 M ferrous (H) chloride and 0.03 M titanium tetrachloride.

### Example 44

Paramagnetic particles of the invention which could be synthesized by co-precipitation method of Example 1 in which the metal salt solution composed of 1.0 M ferrous (H) chloride and 0.01 M titanium tetrachloride.

### Example 45

Paramagnetic particles of the invention which could be synthesized by co-precipitation method of Example 1 in which the metal salt solution composed of 1.0 M ferrous (II) chloride and 0.5 M hafnium chloride.

### Example 46

Paramagnetic particles of the invention which could be synthesized by co-precipitation method of Example 1 in which the metal salt solution composed of 1.0 M ferrous (II) chloride and 0.2 M hafnium chloride.

### Example 47

Paramagnetic particles of the invention which could be synthesized by co-precipitation method of Example 1 in which the metal salt solution composed of 1.0 M ferrous (H) chloride and 0.125 M hafnium chloride.

### Example 48

Paramagnetic particles of the invention which could be synthesized by co-precipitation method of Example 1 in which the metal salt solution composed of 1.0 M ferrous (II) chloride and 0.1 M hafnium chloride.

### Example 49

Paramagnetic particles of the invention which could be synthesized by co-precipitation method of Example 1 in which the metal salt solution composed of 1.0 M ferrous (II) chloride and 0.05 M hafnium chloride.

### Example 50

Paramagnetic particles of the invention which could be synthesized by co-precipitation method of Example 1 in which the metal salt solution composed of 1.0 M ferrous (II) chloride and 0.03 M hafnium chloride.

### Example 51

Paramagnetic particles of the invention which could be synthesized by co-precipitation method of Example 1 in which the metal salt solution composed of 1.0 M ferrous (II) chloride and 0.01 M hafnium chloride.

### Example 33

Paramagnetic particles synthesized by co-precipitation method based on the process of Example 1 with set of acidic metal salt solutions in which 10% to 50% of the volume is ethanol, a propanol, a butanol, ethylene glycol, 1-2-propylene glycol, 1-3-propylene glycol, or mixture thereof.

### Example 34

Paramagnetic particles synthesized by co-precipitation method as described in Example 1 in which the post synthesis buffered solutions are 50 mM to 100 mM solutions composed of Na2 HPO4, Na3PO4, NaBO4, NaCl, acetate salts, or combinations thereof.

### Example 36

A process to coat paramagnetic particles of the invention with silane polymers which would be based on standard chemistry used to silane coat metal oxided surfaces. Examples of silane polymer coatings would be (3-glycidyloxypropyl) trimethoxysilane, 3-aminopropyl trimethoxysilane, 3-thiopropyl trimethoxysilane or the ethoxy silane monomers, mixed with methyl or ethyltrimethoxysilane.

### Example 37

Process based on the paramagnetic particles of the invention as the solid support to purify and concentrating DNA from blood is to add the blood, size range from 1 to 200 μL, to 300 microliters of an extraction buffer composed of 100 mM of N-cyclohexyl-3-aminopropanesulfonic acid (CAPS), 25 mM Na2C03, 2 M Guanidine HCl, 1% sodium lauroyl sarcosine (pH=10.5 to 11) with 2.5 U of alkaline protease (Savinase™). Water is added so that the sample plus extraction buffer volume is 0.5 mL. The sample-extraction buffer mix is incubated for 90 minutes at 55° C. Mixed with the sample extract is added 0.2 mg of the paramagnetic particles of the invention as 20 mg per mL suspension in 100 mM Na2C03. DNA adsorption to the particles is induced by adding LiCl and isopropanol to final concentrations of 290 mM LiCl and 55% (v/v) isopropanol. The paramagnetic particles are collected with a magnetic field and the supernatant is removed. The particles and DNA are washed with 500 microliters of 50% ethanol solution containing 150 mM NaCl, 7.5 mM H3BO4, 25 mM Na2B403, and 0.1% Tween-20. Then a second wash with borate buffered ethanol solution (50% ethanol, 7.5 mM H3BO4, 25 mM Na2B403, 0.1% Tween-20). The twice washed DNA enriched particle pellet are “air dried for ten minutes. The DNA is eluted from the particles with elution buffer composed of 7.5 mM H3BO4, 25 mM Na2B403, and 0.01% Tween-20 and incubated for 15 minutes at 50° C., mixed then incubated for additional 15 minutes at 50° C. The particle pellet was dispersed into this DNA elution buffer and incubated a second time for 15 minutes at 50° C. After DNA elution, the spent particles are removed by magnetic field and the particle free DNA solution is transferred to a new tube.

### Example 38

A DNA isolation process based on Example 37 wherein the DNA precipitation chemistry is 400 mM LiCl, 3% PEG-8,000, and 40% ethanol, or 210 mM LiCl and 57% of 2-propanol.

### Example 40

An RNA isolation process based on the method of Example 37 wherein the extraction solution is 100 nm of 3-morpholinopropane 1-propanesulfonic acid (MOPS), 50 mM Na2C03, 3 M Guanidine Ha, 1% sodium lauroyl sarcosine, with 2.5 U of alkaline protease (Savinase™).

### Example 41

A nucleic acid sequence detection assay that would be based on the sequence specific hybridization to complementary nucleic acids attached or adsorbed to the surface of the paramagnetic particles via silane polymers attached to the surface of the particle.

### Example 42

A nucleic acid sequence detection assay based Example 42 in which the complementary strands is covalently linked to the surface of paramagnetic particles of the invention coated with silane polymer which incorporated 3-aminopropyl ethoxysilane in which the nucleic acid is linked via a vinyl sulfone linkage.

### Example 43

A nucleic acid sequence detection assay based Example 42 in which the complementary strands is covalently linked to the surface of paramagnetic particles of the invention coated with silane polymer which incorporated (3-glycidyloxypropyl) ethoxysilane.

### Example 44

The process for fractionating and concentrating proteins based upon ammonium sulfate precipitation in which the one of the paramagnetic particle of the invention are added to the protein solution at a concentration of 1 milligram per milliliter. The proteins are selectively adsorbed to the particles by the addition of saturated ammonium sulfate to the protein solution with particles in suspension at a concentration that induces the targeted protein to precipitate out of solution. The protein adsorbed to the particles are concentrated by process of magnetic separation and solution is removed from the particle pellet. The adsorbed proteins are eluted from the particles by suspending the particles in a solution that causes the precipitated proteins to dissolve into solution phase.

### Example 45

A process based on enzyme linked immunosorbent assay, i.e. ELISA, in which the solid phase for the immuno-assay are particles of the invention used as colloidal suspensions. Paramagnetic particles of the invention coated with styrene polymer, nitrocellulose, or polymer composed of repeating units of vinyl sulfone linked thioethers, or branched polymers composed of trithiotriazene, triaminotriazene, divinyl sulfone, thioalkyls or thio-alcohols.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way.

The reference numbers recited in the below claims are solely for ease of examination of this patent application, and are exemplary, and are not intended in any way to limit the scope of the claims to the particular features having the corresponding reference numbers in the drawings.

1-30. (canceled)

31. A set of paramagnetic particles synthesized by a co-precipitation methods, the co-precipitation method comprises mixing together an alkaline hydroxide solution and a metal salt solution, in which the metal salt solution comprises at least one ferrous salt and at least one metal salt selected from Group 4 elements of the Periodic Table, wherein the concentration of the ferrous salt is equal to or greater than the concentration of the Group 4 metal salt.

32. The set of paramagnetic particles of claim 31, wherein the Group 4 metal salt comprises a zirconium salt, a titanium salt, a hafnium salt Or mixture of two or more thereof wherein the concentration of the Group 4 metal salt in the metal salt solution is less than or equal to five-tenths the concentration of the ferrous salt.

33. The set of paramagnetic particles of claim 31, wherein the metal salt solution comprised of Group 4 salt and the ferrous salt, and a third metal salt selected from Group 1, Group 2, Group 5, Group 6, Group 7, Group 8, Group 9, Group 10, Group 12 or Group 13 elements of the Periodic Table, in which the concentration of the third metal salt is less than that of the ferrous salt.

34. The set of paramagnetic particles of claim 31, wherein the metal salts that comprise the metal salt solution are selected from but not limited to chloride salts, a sulfate salts, an acetate salts, alkoxy salts, a halide salts, a nitrate salts, or mixtures thereof.

35. The set of paramagnetic particles of claim 33, wherein the metal salts that comprise the metal salt solution are selected from but not limited to chloride salts, a sulfate salts, an acetate salts, alkoxy salts, a halide salts, a nitrate salts, or mixtures thereof.

36. The set of paramagnetic particles of claim 33, wherein the third metal salt comprises a lithium salt at a concentration between one-hundredth to two-tenths the concentration of the ferrous salt.

37. The set of paramagnetic particles of claim 33, wherein the third metal salt comprises a Group 2 metal salt, selected from a magnesium salt, a calcium salt, a strontium salt, a barium salt, or a mixture thereof, in which the total concentration of the Group 2 metal salt, or salt mixture, is between one-hundredth to two-tenths the concentration of the ferrous salt.

38. The set of paramagnetic particles of claim 33, wherein the third metal salt comprises a ferric salt at a concentration between one-hundredth to two-tenths the concentration of the ferrous salt.

39. The set of paramagnetic particles of claim 33, wherein the third metal salt comprises an aluminium salt, a gallium salt, or a mixture thereof, in which the total concentration of the Group 13 metal salt, or salt mixture, is between one-hundredth to two-tenths the concentration of the ferrous salt.

40. The set of paramagnetic particles of claim 33, wherein the third metal salt at a concentration between one-hundredth to one-tenth the concentration of the ferrous salt with the third metal salt selected from a vanadium salt, a manganese salt, a chromium salt, a cobalt salt, a nickel salt, or a zinc salt.

41. The set of paramagnetic particles of claim 31, wherein the alkaline solution comprises of one or a mixture of ammonium hydroxide, sodium hydroxide, potassium hydroxide, urea, or mixtures thereof.

42. The set of paramagnetic particles of claim 33, wherein the paramagnetic particles are treated with a solution comprising:
(a) an anion selected from the group consisting of: acetates, amino acids, peptides, alkoxysilanes, halosilanes, borates, carbonates, carboxylates, citrates, halides, fluoride, perchlorates, phosphates, phosphonates, sulfites, sulfates, and sulfonates;
(b) a glycol selected from the group consisting of glycerol, ethylene glycol, 1,2 propylene glycol, 1,3 propylene glycol, glycerol, 1,2 butylene glycol, 1,3 butylene glycol, 2,3 butylene glycol, polyglycol polymers including polyethylene glycol and polypropylene glycol;
(c) a modified saccharide or derivative thereof selected from the group consisting of mannitol, sorbitol, or erythritol;
(d) a detergent or surfactant selected from the group consisting of dodecyl sulfates, lauroyl sarcosines, poiyoxyethylenesorbitans, oleic acid, palmitic acid, octanoic acid, sulfate detergents, phosphate detergents, zwitterionic detergents, and carbonate detergents.

43. The set of paramagnetic particles of claim 31 with a coating or a set of coatings applied to the particles in which the coating comprising of apatite, carbonate, carbon-based polymer including plastic, metal oxides, silicates, slime or silicon polymers or mixtures thereof.

44. The set of paramagnetic particles of claim 33 with a coating or a set of coatings applied to the particles in which the coating comprising of apatite, carbonate, carbon-based polymer including plastic, metal oxides, silicates, same or silicon polymers or mixtures thereof.

45. The set of paramagnetic particles of claim 44, wherein a silicon-based coatings for the paramagnetic particles are selected composed of but not limited to alkoxysilanes or halosilanes, or mixtures thereof, selected from but not limited to dichlorosilane, trichlorosilane, tetrachlorosilane, methoxysilanes, and ethoxysilanes, aminopropylsilanes, mercaptopropylsilanes, or epoxypropylsilanes, or mixtures thereof.

46. The set of paramagnetic particles of claim 43, wherein the surface of the paramagnetic particles function to bind biomolecules by covalent linkage to an activated moiety linked to the particle surface selected from by not limited to divinyl sulfone, acrylamides, aldehydes, epoxides, thiols, succinamides, vinyls, or mixtures thereof.

47. The set of paramagnetic particles of claim 44, wherein the surface of the paramagnetic particles function to bind biomolecules by covalent linkage to an activated moiety linked to the particle surface selected from by not limited to divinyl sulfone, acrylamides, aldehydes, epoxides, thiols, succinamides, vinyls, or mixtures thereof.

48. The set of paramagnetic particles of claim 33 being part of a kit, wherein the paramagnetic particles are used in processes as the solid phase for separating, isolating, purifying, fractionating, concentrating, or detecting cells, biocomplexes, or biomolecules selected from but not limited to nucleic acids, oligonucleotides, proteins, polypeptides, peptides, carbohydrates, lipids, or combinations thereof.

49. A method for purifying or isolating biological molecules such as proteins or a nucleic acids from a biological source using a solid phase composed of the particles of claim 33 by a protocol that comprises the protein or nucleic acid to partition with solid phase due to surface adsorption or co-aggregation induced by salts, polymers, alcohols, or organic solutions.

50. The method of claim 49 for purifying or isolating a nucleic acid from a biological source based on solid phase chemistry common to the art, in which the solid phase is composed of the particles of claim 33 by a protocol that comprises these with steps:
(a) mixing the biological source of nucleic acid with an extraction buffer comprising a detergent, one chaotropic agent, undo protease enzyme; with the a chaotropic compound selected from but not limited to guanidine salts, urea, formamide, or isothiocyanate salts; a detergent or surfactant selected from but not limited to sodium dodecyl sulfate, N Lauroylsacosine, sodium [dodecanoyl (methyl) amino]acetate, hexadecyltrimethyl-ammonium bromide, t-octylphenoxypolyethoxyethanol, polyoxyethylene (20) sorbitan monooleate, polyoxyethylene (20) sorbitan monolaurate; a protease enzyme selected from but not limited to proteinase K, protease substilisin, and serine type proteases;
(b) adding and dispersing the set of paramagnetic particles of claim 33 to the mixture of step (a) until the set of paramagnetic particles are evenly distributed throughout the solution;
(c) adding a nucleic acid precipitating agent to the mixture of step (b) to a concentration that is sufficient to induce all or a select set of the nucleic acids to partition out of solution phase and to aggregate with or adsorb to the surfaces of the paramagnetic particles with nucleic acid precipitating agent selected from but not limited to ethanol, 2-propanol, polyethylene glycol, guanidine salts, sodium iodide, sodium perchlorate, lithium chloride, hexadecyltrimethylammonium bromide, spermine, spermidine, or mixtures thereof.
(d) concentrating the paramagnetic particles out from suspension phase by the use of a magnetic field to concentrate the particle bound nucleic acids;
(e) removing the liquid phase from the concentrated paramagnetic particles;
(f) suspending the concentrated paramagnetic particles back to suspension phase in a solution that causes the particle-bound nucleic acid to partition to solution phase; and
(g) collecting the paramagnetic particles from suspension by use of a magnetic field and retrieving the solution phase nucleic acid by transfer the solution to a new vessel.
